(19) United States
(12) Patent Application Publication
(10) Pub. No.: US 2022/0361804 A1
(43) Pub. Date: Nov. 17, 2022

(54) INVOLUNTARY HUMAN MOTION ANALYSIS AND DISPLAY

(71) Applicant: Tremor Testing and Data Analytics, Inc., Moorhead, MN (US)

(72) Inventors: James Martodam, Moorhead, MN (US); Gregory Odness, Barnesville, MN (US)

(21) Appl. No.: 17/317,639

(22) Filed: May 11, 2021

(51) Int. Cl. A61B 5/00; A61B 5/11

(52) U.S. Cl. CPC A61B 5/4082; A61B 5/1101; A61B 5/0022; A61B 5/743; A61B 5/7475; A61B 5/6826

(57) ABSTRACT

A system and method are disclosed including receiving, from a sensor, motion data captured from a human, the motion data representative of involuntary human motions. In one embodiment, the method includes storing, in a memory, the motion data captured from the human. In one embodiment, the method includes processing the motion data captured by the human to determine a cycle bandwidth of the involuntary human motions across the motion data. In one embodiment, the method includes outputting the cycle bandwidth of the involuntary human motions across the motion data.

### FIELD

This disclosure relates generally to involuntary human motion. More particularly, this disclosure relates to evaluating and displaying involuntary human motion such as, but not limited to, human tremors.

### BACKGROUND

Human tremors are involuntary, rhythmic muscle contractions that cause shaking movements in one or more parts of the body. In some cases, human tremors are minor and not noticeable by an individual. However, in some cases, for example when an individual has a neurogenerative disease such as, but not limited to, Parkinson's Disease, Essential Tremor, or the like, the human tremors can be serious and debilitating. In serious cases, the human tremors can cause a degradation in the quality of life and interfere with everyday activities. Analysis of human tremors often relies upon subjective analysis by medical professionals.

### SUMMARY

In one embodiment a method includes receiving, from a sensor, motion data captured from a human, the motion data representative of involuntary human motions. In one embodiment, the method includes storing, in a memory, the motion data captured from the human. In one embodiment, the method includes processing the motion data captured by the human to determine a cycle bandwidth of the involuntary human motions across the motion data. In one embodiment, the method includes outputting the cycle bandwidth of the involuntary human motions across the motion data.

In one embodiment, the sensor is electronically communicable with the memory via a network.

In one embodiment, the sensor is part of a computer system and the memory is a part of the computer system.

In one embodiment, the method includes displaying the motion data as captured in a first display region of a graphical user interface.

In one embodiment, the method includes displaying the motion data in a graphical form.

In one embodiment, the method includes displaying the cycle bandwidth of the involuntary human motions across the motion data in a graphical form.

In one embodiment, the processing the motion data captured by the human to determine a cycle bandwidth of the involuntary human motions across the motion data is completed by a server system, and the outputting the cycle bandwidth of the involuntary human motions across the motion data comprises sending the cycle bandwidth of the involuntary human motions across the motion data to a computer system communicable with the server system.

In one embodiment, the method includes displaying the cycle bandwidth of the involuntary human motions across the motion data concurrently with the motion data as captured and the motion data in a graphical form.

In one embodiment, the method includes displaying a first hand and a second hand, the first hand and the second hand being automated to show motion based on the motion data for a time period as selected by a user.

In one embodiment, a system includes a sensor; one or more processors; and memory storing instructions that when executed by the one or more processors, cause the one or more processors to perform operations. In one embodiment, the operations include receiving, from a sensor, motion data captured from a human, the motion data representative of involuntary human motions. In one embodiment, the operations include storing, in a memory, the motion data captured from the human. In one embodiment, the operations include processing the motion data captured by the human to determine a cycle bandwidth of the involuntary human motions across the motion data. In one embodiment, the operations include outputting the cycle bandwidth of the involuntary human motions across the motion data.

In one embodiment, the sensor is one of a 6-axis sensor or a 9-axis sensor.

In one embodiment, the sensor is configured to capture measurements at a frequency of 25 Hz to 400 Hz.

In one embodiment, the sensor is configured to capture measurements at a frequency of 50 Hz to 400 Hz.

In one embodiment, the sensor and the one or more processors are physically separate and electronically communicable.

In one embodiment, the sensor is onboard a computer system including the one or more processors.

In one embodiment, the sensor is configured to be disposed at a location of a user's hand.

In one embodiment, the sensor is included in a ring.

In one embodiment, the system includes a display device, wherein the cycle bandwidth of the involuntary human motions across the motion data is displayed on the display device.

In one embodiment, the cycle bandwidth of the involuntary human motions across the motion data as displayed on the display device includes a representation of typical tremor frequencies for one or more health conditions.

In one embodiment, the outputting the cycle bandwidth of the involuntary human motions across the motion data includes outputting a first cycle bandwidth of the involuntary human motions across first motion data and a second cycle bandwidth of the involuntary human motions across second motion data.

### BRIEF DESCRIPTION OF THE DRAWINGS

References are made to the accompanying drawings that form a part of this disclosure and that illustrate embodiments in which the systems and methods described in this Specification can be practiced.

FIG. 1 is a schematic diagram of an involuntary motion analyzer, according to one embodiment.

FIG. 2 is a schematic diagram of an involuntary motion analyzer, according to one embodiment.

FIG. 3 is a schematic diagram of an involuntary motion analyzer, according to one embodiment.

FIG. 4 is a schematic diagram of a computer system, according to one embodiment.

FIG. 5 is a schematic diagram of an involuntary motion analyzer graphical user interface (GUI), according to one embodiment.

FIG. 6 is a schematic diagram of an involuntary motion analyzer GUI, according to one embodiment.

FIG. 7 is a schematic diagram of an involuntary motion analyzer GUI, according to one embodiment.

FIG. 8 is a schematic diagram of an involuntary motion analyzer GUI, according to one embodiment.

FIG. 9 is a schematic diagram of an involuntary motion analyzer GUI, according to one embodiment.

FIG. 10 is a schematic diagram of an involuntary motion analyzer GUI, according to one embodiment.

Like reference numbers represent the same or similar parts throughout.

### DETAILED DESCRIPTION

Involuntary human motion, such as human tremors, are involuntary, rhythmic muscle contractions that cause shaking movements in one or more parts of the body. In some cases, involuntary human motion is minor and not noticeable by an individual. However, in some cases, for example when an individual has a neurogenerative disease such as, but not limited to, Parkinson's Disease, the involuntary human motion can be serious. In serious cases, the involuntary human motion can cause a degradation in the quality of life and interfere with everyday activities. There are limited tools for understanding, tracking, and treating involuntary human motion.

In one embodiment, the disclosure is directed to a system and method for analyzing and displaying involuntary human motion. In one embodiment, the system utilizes a six axis sensor with wireless communication of data via low power communication protocols. In one embodiment, the system utilizes a nine axis sensor with wireless communication of data via low power communication protocols. It is to be appreciated that other sensors (e.g., having fewer than 6, from 6-9, or greater than 9) may be used within the scope of this disclosure.

The system can utilize motion data from several different sources (to be determined by the end user). In one embodiment, the several different sources can include one or more sensors placed in various locations about the human body. For example, in one embodiment, the one or more sensors can be disposed at a location on the hand, on the wrist, on the arm, on the torso, or other location. This sensor data can be stored on one or more computer or cloud-based sites to later be used to measure the tremor motion. The sensors should be capable of detecting motion in multiple axes (e.g., 9 axis sensor) with acquisition rates of 25 Hz to 400 Hz. In one embodiment, the sensors should be capable of detecting motion in multiple axes with acquisition rates of 50 Hz to 400 Hz. In one embodiment, the sensors should be capable of detecting motion in multiple axes with acquisition rates of 10 Hz to 400 Hz. In one embodiment, the acquisition rates can be greater than 400 Hz.

In one embodiment, the sensor can be disposed on the hand. In one embodiment, the sensor can be disposed on a body part other than the hand. For example, in one embodiment, the one or more sensors can be disposed at a location on the arm, torso, legs, feet, or other location. In one embodiment, multiple sensors can be utilized and disposed at different locations on the body. In one embodiment, the sensor can be embedded into a wearable device such as, but not limited to, a ring, a wristband, or the like. In one embodiment, the sensor can be embedded in a mobile device such as, but not limited to, a smartphone, a smartwatch, or the like.

In one embodiment, tremor motions can be displayed by tremor cycle, tremor cycle bandwidth, or the like. Advantageously, tremor cycles can be more accurate in identifying tremor motion as opposed to current methods which rely upon frequency and peak frequency.

In one embodiment, an inclusion of a (SNR) signal to noise ratio within the calculation of frequency levels can provide the ability to conduct detailed analysis of segmented portions based on seconds of time or as percentage of session data. In one embodiment, a segment can include a minimum threshold of cycles. For example, in one embodiment, a segment can include at least 200 cycles worth of data. In one embodiment, an average number of cycles per segment can be used to identify a motion pattern in the motion data.

As used herein, a “threshold” is an offset from 0 for evaluating a cycle in motion data for a user. In one embodiment, the threshold is not offset from 0 and can be set to 0. In one embodiment, the threshold can be a user-selected value.

As used herein, a “cycle” is a single change from first crossing of a threshold, crossing zero, to a second crossing of the threshold in the same direction. For example, a cycle includes a change crossing the threshold in an upward direction, to below the threshold, across zero, and back above the threshold again. It is to be appreciated that a cycle can be the inverse of the above as well. For example, a cycle includes a change crossing the threshold in a downward direction, to above the threshold, across zero, and back below the threshold again. If there is fluctuation around zero, but without crossing zero, the pattern may not be counted as a cycle. A cycle is different from a peak in the motion data because a cycle is evaluated relative to the threshold.

In one embodiment, an involuntary motion score can be computed. In one embodiment, the score can be based on an x-axis motion. In one embodiment, the score can be based on a y-axis motion. In one embodiment, the score can be based on a combination of the x-axis and the y-axis motion. In one embodiment, a combined score can include an average of the score for the x-axis and the score for the y-axis.

In one embodiment, the score can be based on the cycle bandwidth of a tremor cycle. In one embodiment, the score can be representative of a likelihood that the motion data is representative of a particular motion type, such as, but not limited to, a Parkinson's tremor. In one embodiment, the score can take into account situations where the x-data and the y-data is in-phase. Such motion can be indicative of motion in a z-direction (even when z-direction is not measured). In one embodiment, such in-phase motion data can be representative of rotational motion, which can be referred to as pill rolling, and is typically indicative of a Parkinson's tremor. In one embodiment, the score can be based on a percentage of the motion that is within a particular cycle bandwidth versus a percentage of the motion that is outside that particular cycle bandwidth.

In one embodiment, motion data in an x-axis can be displayed separately from motion data in a y-axis. In one embodiment, motion data can additionally be captured relative to a z-axis. Such motion data can be displayed separately from motion data in the x-axis and motion data in the y-axis. In one embodiment, motion data in the z-axis may not be needed.

In one embodiment, motion data from different samplings can be overlaid for comparative analysis.

In one embodiment, cycle bandwidths from published medical research frequency levels can be displayed by types of tremors. In one embodiment, these labels can simplify an effort in assessing the motion data for a patient and in identifying correlations between data displayed and an individual's medical charts.

In one embodiment, motion data can be captured within a selected period of time and then be repeated to determine patterns in the detected motion. In one embodiment, the analysis of the motion data can be performed in real-time or substantially real-time. In one embodiment, the analysis can be performed for comparisons of data from previous test sessions.

In one embodiment, graphical information representative of the tremors over time can be displayed to enable review of different aspects of the motion data. For example, in one embodiment, the motion data can be displayed in multiple axes relative to the timestamp in the test. In one embodiment, the motion data can display a number of tremors occurring at particular frequencies, enabling a user to determine which frequency of tremor occurs most frequently.

In one embodiment, the systems and methods described herein can be used identifying disease types of a patient (e.g., Parkinson's, Essential Tremor, and other diseases). In one embodiment, the systems and methods described herein can be combined with other disease symptoms and diagnostics in identifying disease types of a patient.

In one embodiment, the motion data can be stored to allow for review and analysis at a later time. In one embodiment, historical data from multiple people or from multiple tests of a single person can be used to characterize the motion relative to various tremor related diseases. In one embodiment, data learned over time can be used to refine characterizations of cycle bandwidths of tremor cycles.

In one embodiment, the sensor and the processor may not be in the same location.

In one embodiment, the sensor and processor may be a single unit (e.g., a mobile device having an application installed thereon, or the like).

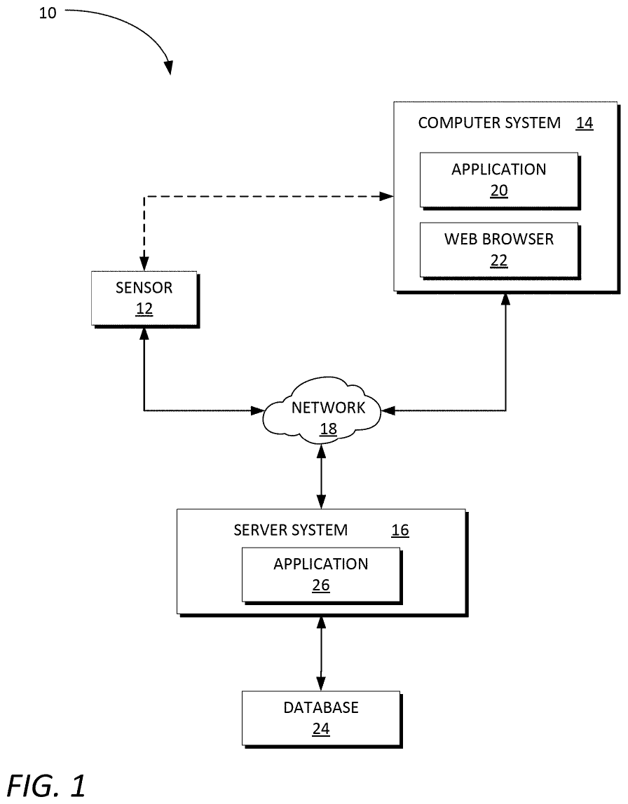

FIG. 1 is a schematic diagram of an involuntary motion analyzer 10, according to one embodiment. The involuntary motion analyzer 10 can generally be used to collect motion data from a human, process the motion data, and present the processed motion data to a user. In one embodiment, the user can be a doctor, nurse, or other health professional. In one embodiment, the user can be an individual other than a doctor (e.g., neurologist or other specialist, or the like), nurse, or health professional. The involuntary motion analyzer 10 can assist with capturing and analyzing involuntary human motion to, for example, determine a severity of the involuntary human motion, determine a treatment plan for the involuntary human motion, or any combination thereof.

The involuntary motion analyzer 10 includes a sensor 12, computer system 14, and server system 16 electronically communicable with one another via a network 18. It is to be appreciated that the illustration is an example and that the involuntary motion analyzer 10 can vary in architecture. Generally, the involuntary motion analyzer 10 can include more than one computer system 14 in communication with the server system 16 via the network 18. In one embodiment, the involuntary motion analyzer 10 can include more than one sensor 12 in communication with the server system 16.

In one embodiment, the computer system 14 includes an application 20 and a web browser 22. In one embodiment, the application 20 or the web browser 22 can be used to analyze and display motion data. In one embodiment, the computer system 14 can receive motion data for display from the server system 16.

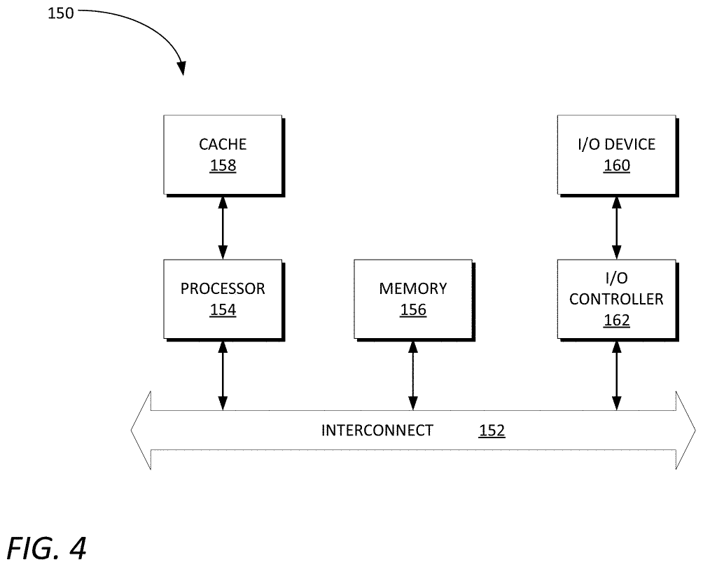

Examples of the computer system 14 include, but are not limited to, a personal computer (PC), a laptop computer, a mobile device (e.g., a smartphone, a personal digital assistant (PDA), a tablet-style device, etc.), a wearable mobile device (e.g., a smart watch, a head wearable device, etc.), or the like. The computer system 14 generally includes a display and an input. Examples of the display for the computer system 14 include, but are not limited to, a monitor connected to a PC, a laptop screen, a mobile device screen, a tablet screen, a wearable mobile device screen, or the like. Examples of the inputs for the computer system 14 include, but are not limited to, a keyboard, a mouse, a trackball, a button, a voice command, a proximity sensor, a touch sensor, an ocular sensing device for determining an input based on eye movements (e.g., scrolling based on an eye movement), suitable combinations thereof, or the like. The computer system 14 can include aspects that are the same as, or similar to, FIG. 4 below.

In one embodiment, the computer system 14 can receive motion data from the sensor 12. In one embodiment, the computer system 14 can receive the motion data from the sensor 12 without the motion data being sent via the network 18. That is, as illustrated by dashed lines in FIG. 1, the sensor 12 can communicate directly with the computer system 14. For example, the sensor 12 can be configured to communicate with the computer system 14 via a near field communication protocol. In one embodiment, the sensor 12 can communicate with the computer system 14 via, for example, a Bluetooth® connection.

In one embodiment, the computer system 14 can send the motion data as received directly from the sensor 12 to the server system 16 for storage, analysis, or any combination thereof. In one embodiment, the computer system 14 can receive the raw motion data and the processed motion data for display on one or more graphical user interfaces (GUIs). In one embodiment, the computer system 14 can process the raw motion data and send the processed motion data to the server system 16. For example, the application 20 can be configured to process the raw motion data and display the processed motion data, the raw motion data, or any combination thereof.

In one embodiment, the sensor 12 can be configured to send the raw motion data to the server system 16 via the network 18. That is, the sensor 12 can either send the raw motion data to the computer system 14 or the server system 16.

The sensor 12 is generally representative of a multi-axis sensor. In one embodiment, the sensor 12 is capable of capturing motion data in 6 axes. In one embodiment, the sensor 12 is capable of capturing motion in 9 axes. In one embodiment, a sampling frequency of the sensor 12 can be up to 400 Hz. In one embodiment, the sampling frequency can be at least 25 Hz. In one embodiment, the sampling frequency can be greater than 400 Hz.

The sensor 12 can be specifically configured to be worn on a selected location of the body. For example, in one embodiment, the sensor 12 can be configured as a ring to be worn on a finger of the user. In one embodiment, the ring can be configured to be worn on a middle finger of the user. In one embodiment, the ring can be adjustable so that a user can wear the ring on different fingers and so that users having different finger sizes can wear the ring. In one embodiment, the sensor 12 can be configured to be worn on a different body part than the fingers. In one embodiment, the sensor 12 can be configured to be removably secured to the user. For example, the sensor 12 can include an adhesive backing that can be secured to a skin of the user. In one embodiment, the sensor 12 can be configured with adjustable straps or the like for securing to an individual.

In one embodiment, the computer system 14 can receive raw motion data from the server system 16. In such an embodiment, the computer system 14 can receive the raw motion data and process the raw motion data for display on one or more GUIs. In one embodiment, the computer system 14 can receive processed motion data from the server system 16. In such an embodiment, the computer system 14 can display the processed data via one or more GUIs.

The server system 16 can include aspects that are the same as or similar to aspects of FIG. 4 below.

In one embodiment, the network 18 can be representative of the Internet. In one embodiment, the network 18 can include a local area network (LAN), a wide area network (WAN), a wireless network, a cellular data network, combinations thereof, or the like.

The server system 16 can be in electronic communication with a database 24. The database 24 can include, among other features, motion data (e.g., for training a neural network). In one embodiment, the database 24 can include additional information such as, but not limited to, user login information or the like for accessing the application 20 or using the web browser 22.

The server system 16 can include an application 26 configured to process raw motion data received from either the sensor 12 or the computer system 14 and to send the processed motion data to the computer system 14 for display via the application 20 or the web browser 22. In one embodiment, the application 26 can process the raw motion data based broken down by segments. The segments can be used to identify motion patterns that may otherwise not be noticeable based on variation in the raw motion data. For example, the raw motion data may show limited motion in some parts of the raw motion data and larger motion in other parts of the raw motion data. However, breaking the data down into segments may enable determination that similar motion patterns are happening in each portion of the raw motion data, but the significance of the motion is increasing. That is, there may be an almost constant underlying involuntary human motion that varies in significance over time, but the motion may be present throughout an entirety of the raw motion data. This can be made visible by segmenting the raw motion data into a subset of cycles.

It is to be appreciated that the various roles of the server system 16 and the database 24 can be distributed among the devices in the involuntary motion analyzer 10. In one embodiment, the database 24 can be maintained on the server system 16.

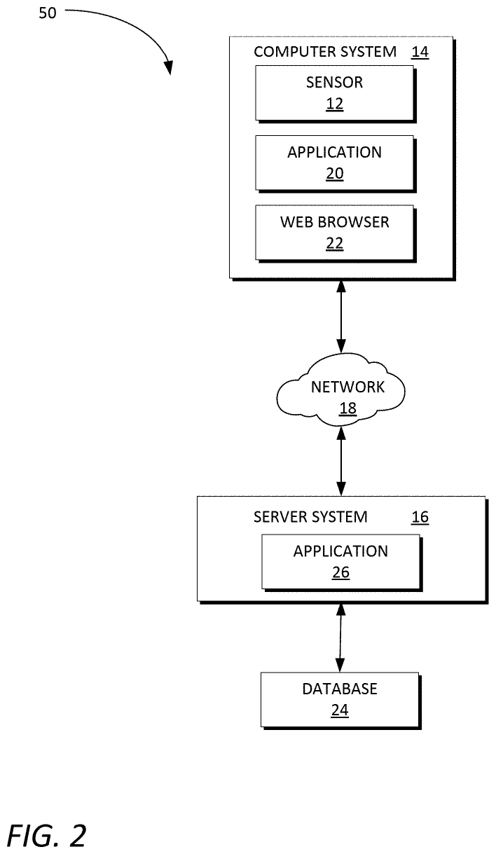

FIG. 2 is a schematic diagram of an involuntary motion analyzer 50, according to one embodiment. The involuntary motion analyzer 50 can generally be used to collect motion data from a human, process the motion data, and present the processed motion data to a user. In one embodiment, the user can be a doctor, nurse, or other health professional. In one embodiment, the user can be an individual other than a doctor, nurse, or health professional. The involuntary motion analyzer 50 can assist with capturing and analyzing involuntary human motion to, for example, determine a severity of the involuntary human motion, determine a treatment plan for the involuntary human motion, or any combination thereof.

For simplicity of this disclosure, aspects of FIG. 2 which are the same as aspects of the involuntary motion analyzer 10 of FIG. 1 are not described again in additional detail.

The involuntary motion analyzer 50 includes the computer system 14 and the server system 16 electronically communicable via the network 18.

The involuntary motion analyzer 50 varies from the involuntary motion analyzer 10 in that the involuntary motion analyzer 50 includes the sensor 12 incorporated into the computer system 14. As a result, the computer system 14 serves to capture the motion data and to display the raw motion data, the processed motion data, or any combination thereof, to the user.

In one embodiment, the computer system 14 can be, for example, a smartphone or the like. In one embodiment, the computer system 14 can be a specially configured device that is provided to an individual to hold during the duration of a test. The same device can then be used by a doctor, nurse, or other medical professional to evaluate the results. In one embodiment, the involuntary motion analyzer 50 can be relatively simpler than the involuntary motion analyzer 10 (FIG. 1) due to a reduction in the number of parts.

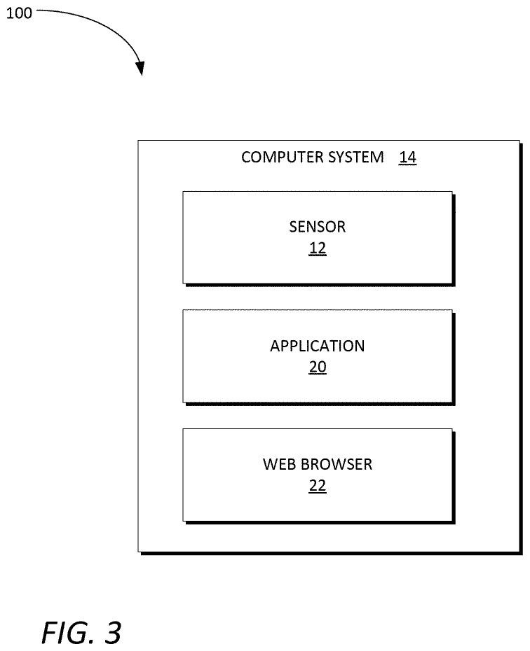

FIG. 3 is a schematic diagram of an involuntary motion analyzer 100, according to one embodiment. The involuntary motion analyzer 50 can generally be used to collect motion data from a human, process the motion data, and present the processed motion data to a user. In one embodiment, the user can be a doctor, nurse, or other health professional. In one embodiment, the user can be an individual other than a doctor, nurse, or health professional. The involuntary motion analyzer 50 can assist with capturing and analyzing involuntary human motions to, for example, determine a severity of the involuntary human motion, determine a treatment plan for the involuntary human motion, or any combination thereof. For simplicity of this disclosure, aspects of FIG. 3 which are the same as aspects of the involuntary motion analyzer 10 of FIG. 1 or the involuntary motion analyzer 50 of FIG. 2 are not described again in additional detail.

The involuntary motion analyzer 100 includes the computer system 14.

The involuntary motion analyzer 100, like the involuntary motion analyzer 50 (FIG. 2) utilizes the sensor 12 that is onboard the computer system 14. That is, the involuntary motion analyzer 100 does not include a separate sensor 12 (FIG. 1). Additionally, the involuntary motion analyzer 100 does not include a server system (e.g., server system 16 (FIG. 1, FIG. 2) is not present in the involuntary motion analyzer 100). As such, the computer system 14 is configured to capture the motion data (e.g., via the sensor 12 that is onboard the computer system 14) and process the raw motion data. The raw motion data is not sent to another system (e.g., the server system 16 FIG. 1, FIG. 2). In one embodiment, the involuntary motion analyzer 100 can be relatively simpler than the involuntary motion analyzer 10 or the involuntary motion analyzer 50, as there is a reduction in the components. In one embodiment, because the computer system 14 is processing health data related to a user, there is a reduced risk of personal information being compromised because the computer system 14 does not provide any of the motion data via a network to another system.

FIG. 4 is a schematic diagram of a computer system 150, according to one embodiment. The computer system 150 can be used to implement the systems and methods of this disclosure. While FIG. 4 illustrates various components of a computer system, FIG. 4 is not intended to represent any particular architecture or manner of interconnecting the components. Other systems that have fewer, or additional, components than shown in FIG. 4 can also be used.

The computer system 150 includes an interconnect 152 that interconnects a processor 154 and a memory 156. The interconnect 152 can include a bus and system core logic, in one embodiment. The processor 154 is coupled to cache memory 158 in the illustrated embodiment.

The interconnect 152 interconnects the processor 154 and the memory 156 and also interconnects the processor 154 and the memory 156 to input/output (I/O) device 160 via an I/O controller 162. The I/O device 160 can include a variety of devices including, but not limited to, a display device, a peripheral device (e.g., a mouse, a keyboard, a modem, a network interface, a printer, a scanner, a video camera, a sensor, any combination thereof, or the like). In one embodiment, the computer system 150 can be a server system, in which case, the I/O device 160 can be optional.

The interconnect 152 can include one or more buses connected to one another through various bridges, controllers, adapters, or any combination thereof. For example, the I/O controller 162 can include a Universal Serial Bus (USB) adapter for controlling USB peripheral devices, an IEEE-1394 bus adapter for controlling IEEE-1394 peripheral devices, or any combination thereof.

The memory 156 can include one or more of read-only memory (ROM), volatile random access memory (RAM), non-volatile memory (e.g., a hard drive, flash memory, or the like).

Volatile RAM can be implemented as dynamic RAM (DRAM) which requires power continually in order to refresh or maintain the data in the memory. Non-volatile memory can include a magnetic hard drive, a magnetic optical drive, an optical drive (e.g., a DVD RAM), or other type of memory system which maintains data even after power is removed from the system. The non-volatile memory may also be a random access memory.

The non-volatile memory can be a local device coupled directly to the rest of the components in the computer system 150. A non-volatile memory that is remote from the system, such as a network storage device coupled to the data processing system through a network interface such as a modem or Ethernet interface, can also be used.

In this description, some functions and operations are described as being performed by or caused by software code to simplify description. However, such expressions are also used to specify that the functions result from execution of the code/instructions by a processor, such as a microprocessor.

Alternatively, or in combination, the functions and operations as described here can be implemented using special purpose circuitry, with or without software instructions, such as using Application-Specific Integrated Circuit (ASIC) or Field-Programmable Gate Array (FPGA). Embodiments can be implemented using hardwired circuitry without software instructions, or in combination with software instructions. Thus, the techniques are limited neither to any specific combination of hardware circuitry and software, nor to any particular source for the instructions executed by the data processing system.

While one embodiment can be implemented in fully functioning computers and computer systems, various embodiments are capable of being distributed as a computing product in a variety of forms and are capable of being applied regardless of the particular type of machine or computer-readable media used to actually effect the distribution.

At least some aspects disclosed can be embodied, at least in part, in software. That is, the techniques may be carried out in a computer system or other data processing system in response to its processor, such as a microprocessor, executing sequences of instructions contained in a memory, such as ROM, volatile RAM, non-volatile memory, cache or a remote storage device.

Routines executed to implement the embodiments may be implemented as part of an operating system or a specific application, component, program, object, module or sequence of instructions referred to as “computer programs.” The computer programs typically include one or more instructions set at various times in various memory and storage devices in a computer, and that, when read and executed by one or more processors in a computer, cause the computer to perform operations necessary to execute elements involving the various aspects.

A machine readable medium can be used to store software and data which when executed by a data processing system causes the system to perform various methods. The executable software and data may be stored in various places including for example ROM, volatile RAM, non-volatile memory and/or cache. Portions of this software and/or data may be stored in any one of these storage devices. Further, the data and instructions can be obtained from centralized servers or peer to peer networks. Different portions of the data and instructions can be obtained from different centralized servers and/or peer to peer networks at different times and in different communication sessions or in a same communication session. The data and instructions can be obtained in entirety prior to the execution of the applications. Alternatively, portions of the data and instructions can be obtained dynamically, just in time, when needed for execution. Thus, it is not required that the data and instructions be on a machine readable medium in entirety at a particular instance of time.

Examples of computer-readable media include but are not limited to recordable and non-recordable type media such as volatile and non-volatile memory devices, read only memory (ROM), random access memory (RAM), flash memory devices, floppy and other removable disks, magnetic disk storage media, optical storage media (e.g., Compact Disk Read-Only Memory (CD ROM), Digital Versatile Disks (DVDs), etc.), among others. The computer-readable media may store the instructions.

The instructions may also be embodied in digital and analog communication links for electrical, optical, acoustical or other forms of propagated signals, such as carrier waves, infrared signals, digital signals, etc. However, propagated signals, such as carrier waves, infrared signals, digital signals, etc. are not tangible machine readable medium and are not configured to store instructions.

In general, a machine readable medium includes any mechanism that provides (i.e., stores and/or transmits) information in a form accessible by a machine (e.g., a computer, network device, personal digital assistant, manufacturing tool, any device with a set of one or more processors, etc.).

In various embodiments, hardwired circuitry may be used in combination with software instructions to implement the techniques. Thus, the techniques are neither limited to any specific combination of hardware circuitry and software nor to any particular source for the instructions executed by the data processing system.

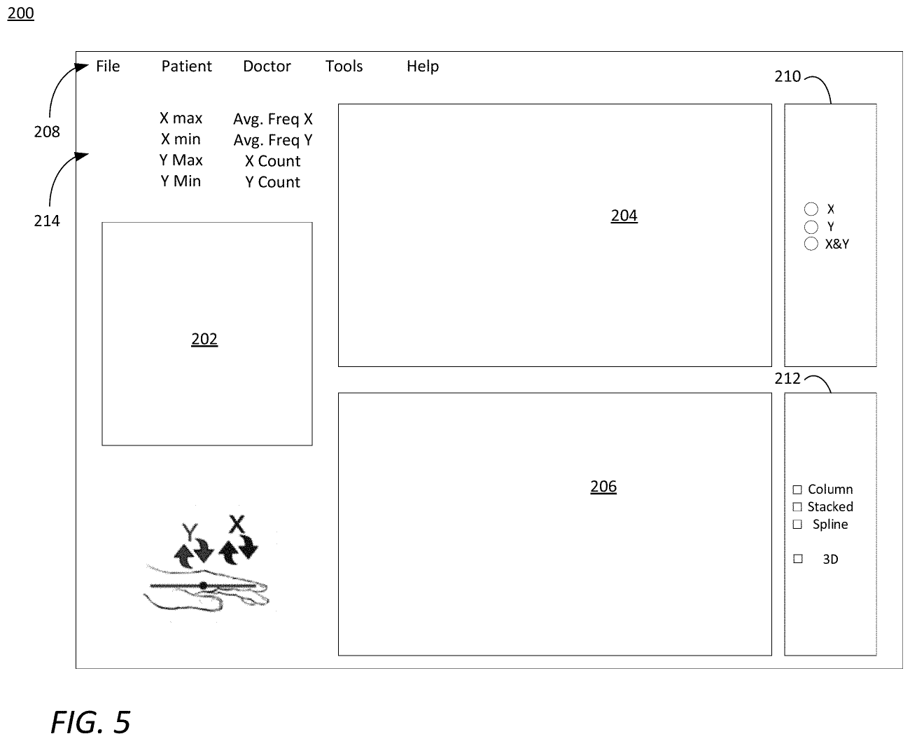

FIG. 5 is a schematic diagram of an involuntary motion analyzer graphical user interface (GUI) 200, according to one embodiment. The GUI 200 can be, for example, representative of a startup screen when using an involuntary motion analyzer (e.g., involuntary motion analyzer 10 (FIG. 1), involuntary motion analyzer 50 (FIG. 2), or involuntary motion analyzer 100 (FIG. 3)). That is, the GUI 200 is representative of the involuntary motion analyzer having no motion data loaded.

The GUI 200 has three primary display regions.

A first display region 202 can be utilized for displaying the raw motion data (e.g., as captured via the sensor 12 (FIGS. 1-3)).

A second display region 204 can be utilized for displaying the motion data based on a timestamp (x-axis in the plot) and displacement (y-axis in the plot). One or more values in the raw motion data can be highlighted (e.g., color coded or the like) to identify a point of interest (e.g., a maximum, a minimum, or the like).

A third display region 206 can be utilized for displaying a cycle bandwidth summary that shows a cycle bandwidth of tremors for a particular cycle.

As such, the GUI 200 can provide several ways of visualizing the motion data—in its raw form, in a graphical form, and in a graphical summary form. The various levels of display of the motion data can, for example, enable a doctor, nurse, or other healthcare professional to efficiently review a user's motion data. No data has been loaded into the GUI 200, so the first display region 202, the second display region 204, and the third display region 206 are shown as blank spaces.

The GUI 200 includes a menu bar 208 including a plurality of menus that the user can use to, for example, load motion data, save the user's analyses, export motion data, any combination thereof, or the like.

The second display region 204 includes a menu 210 including a plurality of options for manipulating the view of the motion data in the second display region 204.

The third display region 206 includes a menu 212 including a plurality of options for manipulating the view of the summary data in the third display region 206.

The GUI 200 additionally includes a data summary region 214. The data summary region 214 provides highlighted portions of the data from the first display region 202. For example, the data summary region 214 can provide a snapshot that includes, for example, an x maximum and minimum value, a y minimum and maximum value, an average frequency for the x motion data, an average frequency for the y motion data, x and y counts, an x average peak amplitude and a y average peak amplitude. The data summary region 214 can be updated and change as the second display region 204 is manipulated by the user.

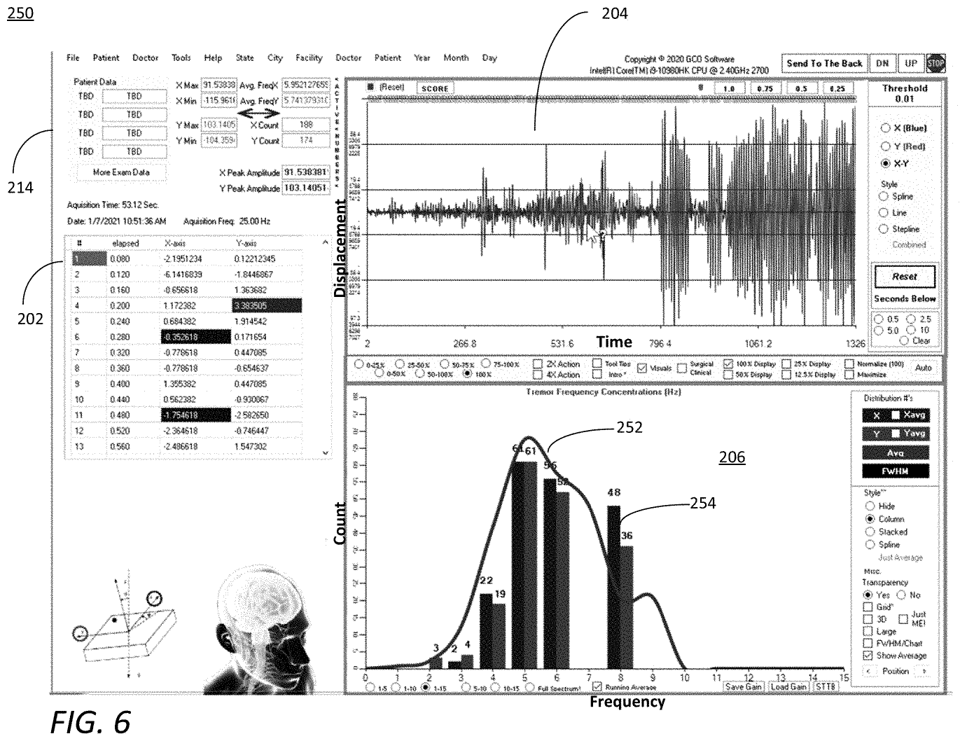

FIG. 6 is a schematic diagram of an involuntary motion analyzer GUI 250, according to one embodiment. For simplicity of this disclosure, features described respective of FIG. 5 will not be described in additional detail.

The GUI 250 includes loaded motion data. The raw motion data is being displayed in the first display region 202. Motion over time is plotted in the second display region 204. Tremor summary data is plotted in the third display region 206. As can be seen in the third display region 206, a combination of a trend line 252 and individual tremor cycle bandwidth bars 254 are shown for efficient reviewing of the motion data as selected. The user is able to manipulate the plots in the second display region 204 and the third display region 206 to, for example, zoom in on particular locations within the motion data, display stepwise curves (instead of smooth curves), focus on only x-axis motion data instead of y-axis motion data, any combination thereof, or the like. The user may also be able to manipulate the plots to show in different formats (e.g., as a 3-D bar chart or the like).

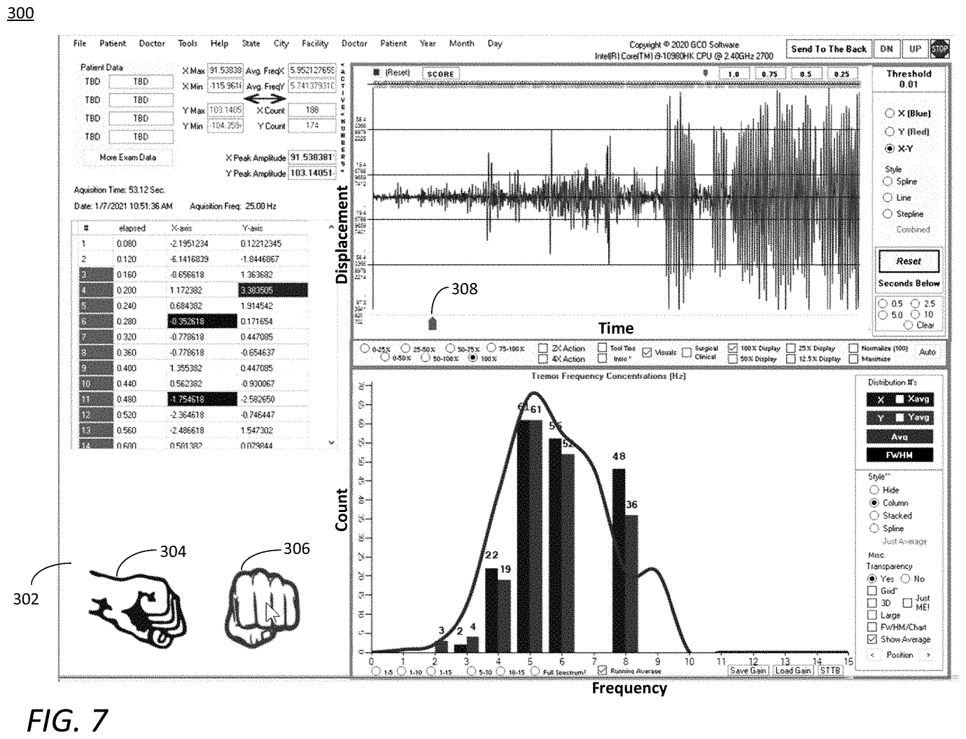

FIG. 7 is a schematic diagram of an involuntary motion analyzer GUI 300, according to one embodiment.

For simplicity of this disclosure, features described respective of FIGS. 5-6 will not be described in additional detail.

In the GUI 300, a fourth display region 302 is shown including two images. In the illustrated embodiment, the two images are two views of a hand. The two views of the hand in the fourth display region 302 correspond to an axis of measurement. For example, hand 304 is representative of x-axis motion and hand 306 is representative of y-axis motion. As illustrated, the axes of motion are perpendicular to each other. In the GUI 300, a tracker button 308 is displayed in the second display region 204. The tracker button 308 can be moved left-right (with respect to the page) to animate the hand 304 and the hand 306 based on the motion data at the selected time. Accordingly, the user can playback the motion data over time while viewing movement of the hand 304 and the hand 306. The automation can help demonstrate a significance of the motion over time. Instead of moving the tracker button 308, the user can select a time period and the GUI 300 can play the entire time period back for the user.

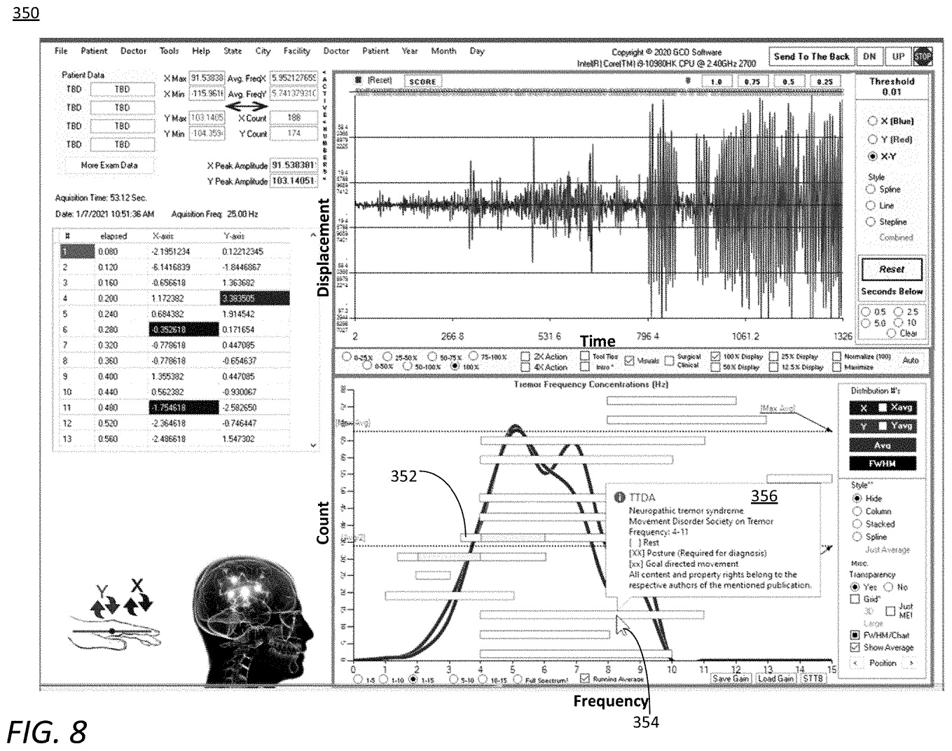

FIG. 8 is a schematic diagram of an involuntary motion analyzer GUI 350, according to one embodiment.

For simplicity of this disclosure, features described respective of FIGS. 5-7 will not be described in additional detail.

In the GUI 350, the third display region 206 is modified to include predefined involuntary human motion classification ranges 352. The user can toggle the display of the predefined involuntary human motion classification ranges 352 to provide some indication of how the displayed tremor summary relates to the predefined involuntary human motion classification ranges 352. When the predefined involuntary human motion classification ranges 352 are displayed, on hovering a mouse indicator 354 over a particular one of the predefined involuntary human motion classification ranges 352, a popup message 356 can be automatically displayed to provide identifying information about the predefined involuntary human motion classification ranges 352. In this manner, a user can efficiently categorize the tremor summary data within one of the predefined involuntary human motion classification ranges 352. In one embodiment, the predefined involuntary human motion classification ranges 352 are based on, for example, published scientific research. In one embodiment, the predefined involuntary human motion classification ranges 352 can be modified over time based on the historical data captured by the involuntary motion analyzer. As a result, the real-world data collected from different users can be used to better define the predefined involuntary human motion classification ranges 352 relative to real-world conditions. Additionally, as shown in FIG. 8, multiple sets of motion data can be displayed at the same time. As a result, two tremor summary lines are plotted in the third display region 206. Plotting multiple data sets at the same time can allow for analysis of motion data under different conditions, under the same conditions at different times, before and after a change in medication to treat the tremors, or the like.

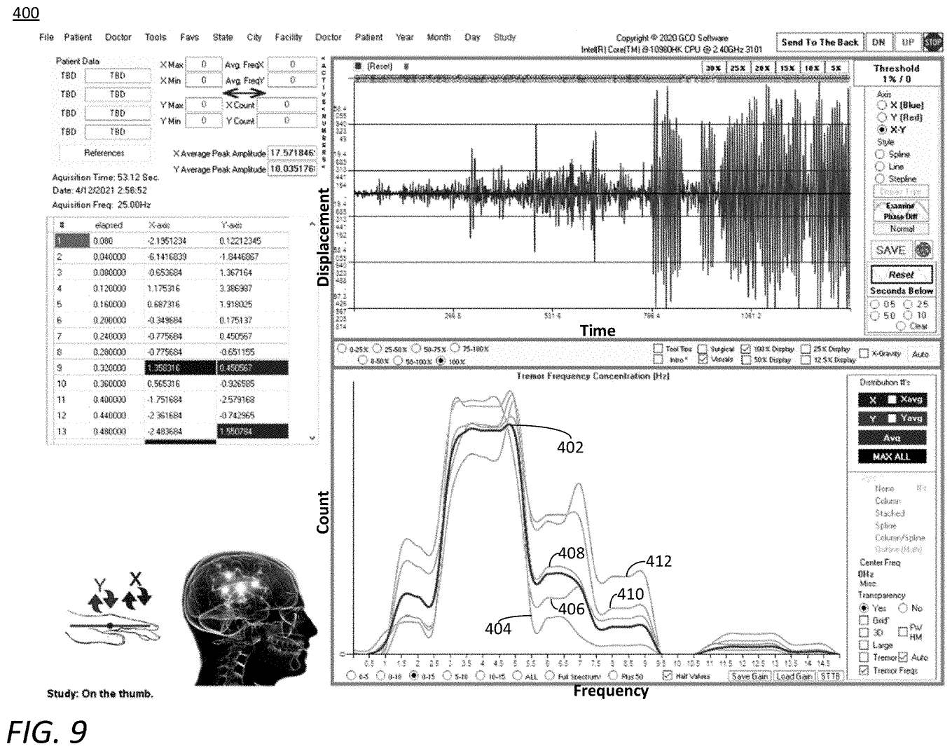

FIG. 9 is a schematic diagram of an involuntary motion analyzer GUI 400, according to one embodiment.

In the illustrated embodiment, a plurality of cycles of data are shown in the third display region 206. The line 402 can be representative of the entire motion data, where each of the lines 404-412 are representative of each of a plurality of segments of the raw motion data. As is visible, a general pattern of the cycle bandwidth is similar across the segments and the entire motion data. In one embodiment, the GUI 400 may enable a better understanding of the involuntary motion of the individual, which is present regardless of the segment, even though a magnitude of the motion over time changes (as shown in the second display region 204).

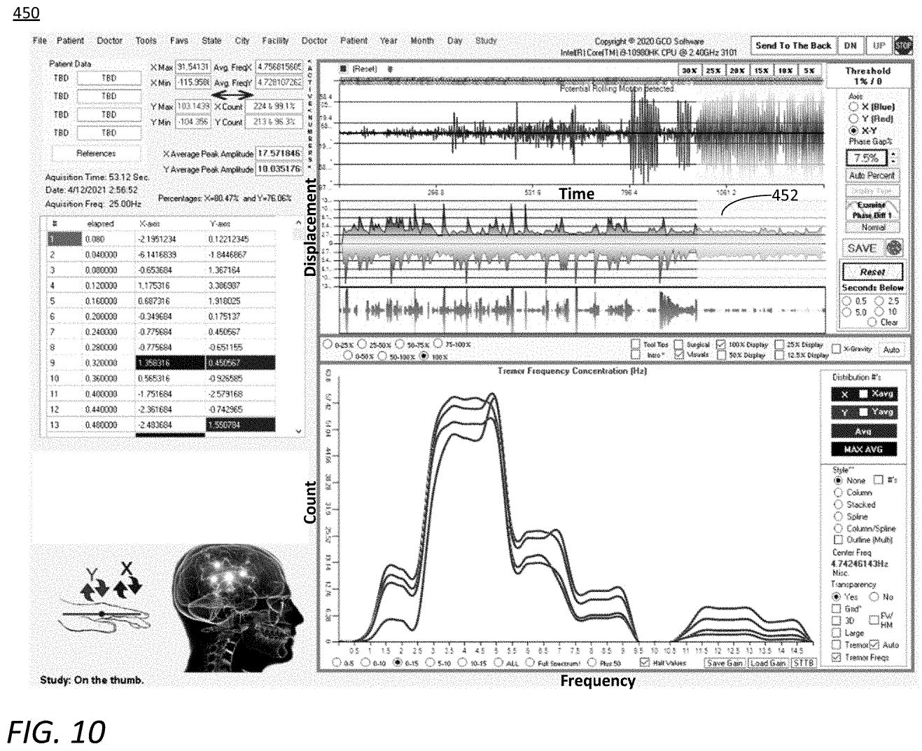

FIG. 10 is a schematic diagram of an involuntary motion analyzer GUI 450, according to one embodiment.

As discussed above, the raw motion data can include data relative to an x-axis and a y-axis. In some cases, motion in the x-axis and the y-axis can be in phase. Such motion can be representative of motion relative to a z-axis. As shown in GUI 450, such in-phase motion can be illustrated in the second display region 204 by showing the x-axis data and the y-axis data separated from each other in phase display 452.

The terminology used herein is intended to describe embodiments and is not intended to be limiting. The terms “a,” “an,” and “the” include the plural forms as well, unless clearly indicated otherwise. The terms “comprises” and/or “comprising,” when used in this Specification, specify the presence of the stated features, integers, steps, operations, elements, and/or components, but do not preclude the presence or addition of one or more other features, integers, steps, operations, elements, and/or components.

It is to be understood that changes may be made in detail, especially in matters of the construction materials employed and the shape, size, and arrangement of parts without departing from the scope of the present disclosure. This Specification and the embodiments described are examples, with the true scope and spirit of the disclosure being indicated by the claims that follow.

1. A method, comprising:
obtaining, by one of a 6-axis sensor or a 9-axis sensor, motion data captured from a human,
wherein the motion data is representative of involuntary human motions,
wherein the motion data includes displacement data of the sensor in at least an x-axis and a y-axis,
wherein the one of the 6-axis sensor or the 9-axis sensor is configured to be attached to a body portion of the human;
transmitting, by the one of the 6-axis sensor or the 9-axis sensor, the motion data wirelessly to a processor via a network;
receiving, from one of the 6-axis sensor or the 9-axis sensor, the motion data captured from the human and wirelessly transmitted to the processor via the network, the motion data including the displacement data of the sensor in at least the x-axis and the y-axis;
storing, in a memory, the motion data as received from the one of the 6-axis sensor or the 9-axis sensor;
processing, by the processor, the motion data captured as received from the one of the 6-axis sensor or the 9-axis sensor to determine a cycle of the involuntary human motions across the motion data,
wherein the cycle of the involuntary human motions across the motion data includes a change in the displacement data in a direction of at least one of the x-axis or the y-axis from a first crossing of a threshold in a first direction, crossing zero in a second direction, to a second crossing of the threshold in the first direction, wherein the threshold is an offset from zero in the direction of at least one of the x-axis or the y-axis;
outputting the cycle of the involuntary human motions across the motion data as determined; and
displaying, by a display device, a number of the cycles at a particular frequency of the involuntary human motions across the motion data as output in a graphical form.

2. (canceled)

3. The method of claim 1, wherein the one of a 6-axis sensor or a 9-axis sensor is part of a computer system and the memory is a part of the computer system.

4. The method of claim 1, wherein the displaying, by the display device, comprises displaying the motion data as captured in a first display region of a graphical user interface.

5. The method of claim 1, wherein the displaying, by the display device, comprises displaying the motion data in a graphical form.

6. (canceled)

7. The method of claim 1, wherein the processing the motion data captured as received from the one of the 6-axis sensor or the 9-axis sensor to determine a cycle of the involuntary human motions across the motion data is completed by a server system, and the outputting the cycle of the involuntary human motions across the motion data as determined comprises sending the cycle of the involuntary human motions across the motion data to a computer system communicable with the server system.

8. The method of claim 1, wherein the displaying, by the display device, comprises displaying the cycle of the involuntary human motions across the motion data concurrently with the motion data as captured in a graphical form.

9. The method of claim 1, comprising displaying a first hand and a second hand, the first hand and the second hand being automated to show motion based on the motion data for a time period as selected by the human.

10. A system, comprising:
a sensor,
wherein the sensor is one of a 6-axis sensor or a 9-axis sensor,
wherein the sensor is configured to be attached to a body portion of a human;
one or more processors in communication with the sensor via a network;
memory storing instructions that when executed by the one or more processors, cause the one or more processors to perform operations comprising:
receiving, from the sensor, motion data captured from the human and transmitted from the sensor to the one or more processors via the network,
wherein the motion data captured from the human and transmitted from the sensor to the one or more processors is representative of involuntary human motions,
wherein the motion data includes displacement data of the sensor in at least an x-axis and a y-axis;
storing, in a memory, the motion data captured and received from the one of the 6-axis sensor to the 9-axis sensor;
processing, by the one or more processors, the motion data captured as received from the one of the 6-axis sensor or the 9-axis sensor to determine a cycle of the involuntary human motions across the motion data,
wherein the cycle of the involuntary human motions across the motion data includes a change in the displacement data in a direction of at least one of the x-axis or the y-axis from a first crossing of a threshold in a first direction, crossing zero in a second direction, to a second crossing of the threshold in the first direction, wherein the threshold is an offset from zero in the direction of at least one of the x-axis or the y-axis;
outputting the cycle of the involuntary human motions across the motion data as determined; and
causing display, by a display device, a number of the cycles at a particular frequency of the involuntary human motions across the motion data as output in a graphical form.

11. (canceled)

12. The system of claim 10, wherein the sensor is configured to capture measurements at a frequency of 25 Hz to 400 Hz.

13. The system of claim 10, wherein the sensor is configured to capture measurements at a frequency of 50 Hz to 400 Hz.

14. The system of claim 10, wherein the sensor and the one or more processors are physically separate and electronically communicable.

15. The system of claim 10, wherein the sensor is onboard a computer system including the one or more processors.

16. The system of claim 10, wherein the sensor is configured to be disposed at a location of the human's hand.

17. The system of claim 10, wherein the sensor is included in a ring.

18. (canceled)

19. The system of claim 10, wherein the cycle of the involuntary human motions across the motion data as displayed on the display device includes a representation of typical tremor frequencies for one or more health conditions.

20. The system of claim 10, wherein the outputting the cycle of the involuntary human motions across the motion data as determined includes outputting a first cycle of the involuntary human motions across first motion data and a second cycle of the involuntary human motions across second motion data.

21. The method of claim 1, comprising computing an involuntary motion score based on the cycle of the involuntary human motions across the motion data, wherein the motion score is based on a percentage of the motion data that is within a particular cycle of the involuntary human motions across the motion data.

22. The method of claim 1, wherein the sensor is disposed in a ring configured to be worn on a finger of the human.

23. The method of claim 1, comprising segmenting the motion data to focus on a subset of the motion data to identify patterns within the motion data.
